(19) United States
(12) Patent Application Publication
(10) Pub. No.: US 2014/0038959 A1
(43) Pub. Date: Feb. 6, 2014

(54) METHODS OF TREATING TUMORS

(71) Applicant: Genentech, Inc., (US)

(72) Inventors: Klaus P. Hoeflich, Millbrae, CA (US); Hartmut Koeppen, San Mateo, CA (US); Christy Ong, Palo Alto, CA (US)

(73) Assignee: Genentech, Inc., South San Francisco, CA (US)

(21) Appl. No.: 13/890,411

(22) Filed: May 9, 2013

Related U.S. Application Data
(63) Continuation of application No. PCT/EP2011/070008, filed on Feb. 11, 2012.
(60) Provisional application No. 61/523,062, filed on Aug. 12, 2011.

(51) Int. Cl. A61K 31/519; A61K 45/06

(52) U.S. Cl. CPC A61K 31/519; A61K 45/06
USPC 514/234.5; 514/260.1

(57) ABSTRACT

The present invention relates to a method of treating tumors wherein PAK1 is over-expressed or amplified by co-administering a PAK1 inhibitor and a second anti-hyper-proliferative agent

The present invention discloses a method of treating cancerous tumors which amplify or over-express PAK1 by contacting the tumor with a PAK1 inhibitor in combination with a second anti-proliferative agent.

Protein kinases are a family of enzymes that catalyze phosphorylation of the hydroxyl groups of specific tyrosine, serine, or threonine residues in proteins. Typically, such phosphorylation can dramatically change the function of the protein and thus protein kinases can be pivotal in the regulation of a wide variety of cellular process, including metabolism, cell proliferation, cell differentiation, and cell survival. The mechanism of these cellular processes provides a basis for targeting protein kinases to treat disease conditions resulting from or involving disorder of these cellular processes. Examples of such diseases include, but are not limited to, cancer and diabetes.

Protein kinases can be broken into two types, protein tyrosine kinases (PTKs) and serine-threonine kinases (STKs). Both PTKs and STKs can be receptor protein kinases or non-receptor protein kinases. PAK is a family of non-receptor STKs. The p21-activated protein kinase (PAK) family of serine/threonine protein kinases plays important roles in cytoskeletal organization, cellular morphogenesis, cellular processes and cell survival (Daniels et al., Trends Biochem. Sci. 1999 24: 350-355; Sells et al., Trends Cell. Biol. 1997 7: 162-167). The PAK family consists of six members subdivided into two groups: PAK 1-3 (group I) and PAK 4-6 (group II) which are distinguished based upon sequence homologies and the presence of an autoinhibitory region in group I PAKs. p21-Activated kinases (PAKs) serve as important mediators of Rac and Cdc42 GTPase function as well as pathways required for Ras-driven tumorigenesis. (Manser et al., Nature 1994 367:40-46; B Dummler et al., Cancer Metathesis Rev. 2009 28:51-63; R. Kumar et al., Nature Rev. Cancer 2006 6:459-473).

The present invention relates to methods of treating tumors or hyper-proliferative conditions wherein the tumor cells or hyper-proliferating cells over-express or amplify PAK1 by treating the patient or contacting the tumor with a PAK1 inhibitor and a second anti-hyper-proliferative or anti-tumor agent selected from an inhibitor of EGFR, the Raf/MEK/ERK pathway, Src, Akt or an inhibitor of apoptosis proteins

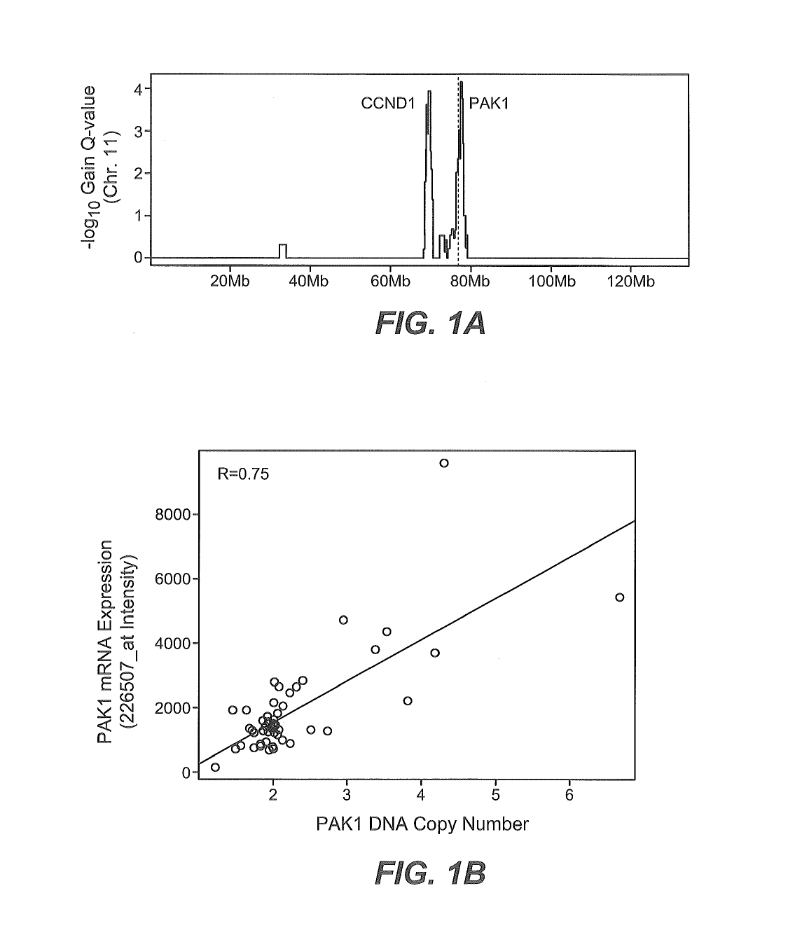

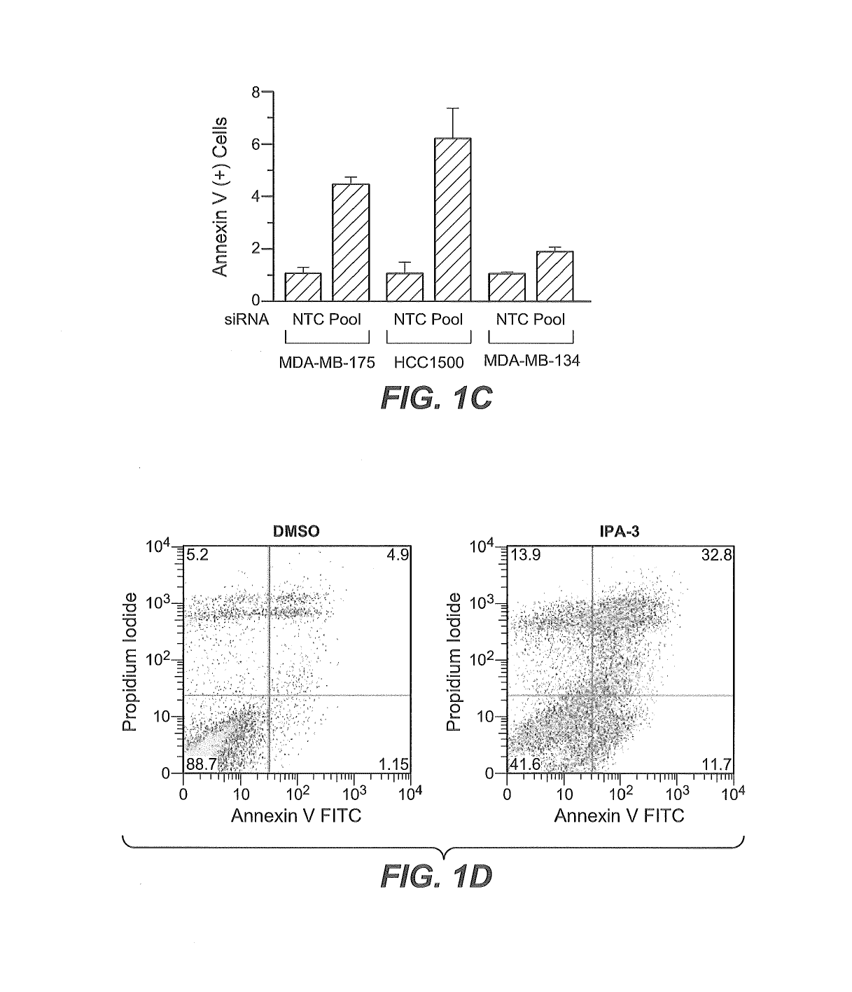

FIG. 1-Analysis of PAK1 genomic amplification and functional role in human breast tumors. (A) Genomic Identification of Significant Targets in Cancer (GISTIC) analysis of 11q13 copy number gains. Points are proportionately spaced and arranged in genome order. Vertical line represents chromosome location of the PAK1 gene. GISTIC Q-value for DNA gain are defined by the multiple-testing-corrected probability of gain frequency and mean copy gain occurring by chance displayed as the negative log₁₀ of the Q-value for each SNP array probe set. (B) PAK1 DNA copy and mRNA expression dot plot depicts the relationship of DNA copy number to the 226507 at Affymetrix MAS 5.0 signal for 51 tumor samples. The Pearson correlation statistic (0.75) is shown for the plot. The solid line represents the best-fit line through these points. (C) Increasing proportion of Annexin V-positive cells following knockdown of PAK1 expression is shown for 3 breast cancer cell lines with focal PAK1 genomic amplification, MDA-MB-175, HCC1500 and MDA-MB-134 IV. Cells were harvested 3-5 days following transient transfection of pooled siRNA oligonucleotides. (D) Fluorescence-activated cell sorting analysis for Annexin V/PI. MDA-MB-175 cells were cultured in the presence or absence of IPA-3 for 48 h. Annexin V/PI staining was then done to assess apoptosis/necrosis. Annexin V labeling (bottom right quadrants) represents the population undergoing early apoptosis. Annexin V and PI double labeling (top right quadrants) represent cells that have already died by apoptosis. Live cells are represented in the bottom left quadrants. Percentages of cells are shown for each quadrant.

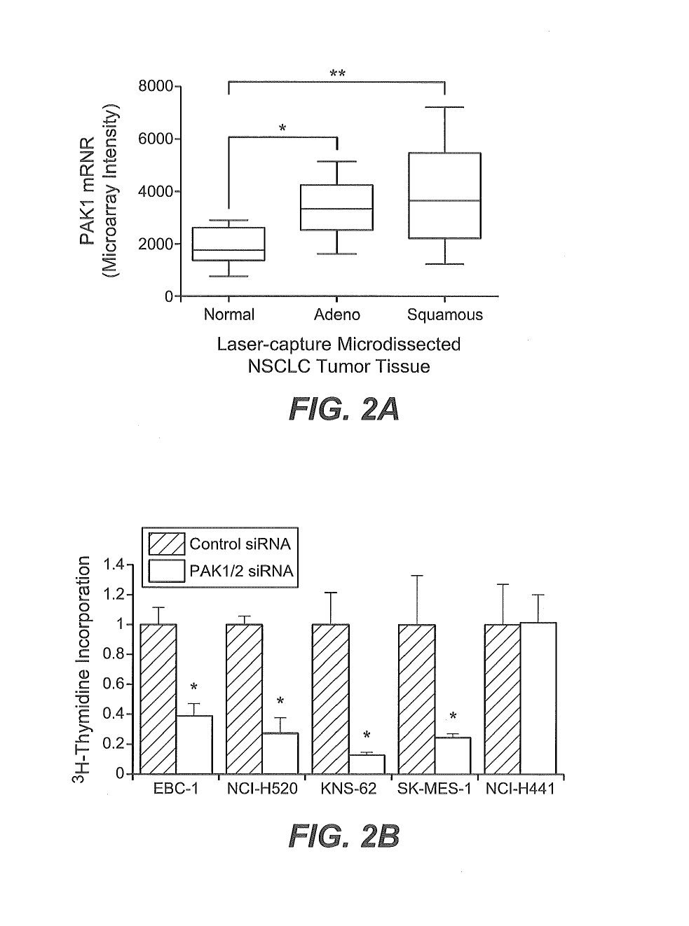

FIG. 2—PAK1 is highly expressed in human lung tumors and plays a critical role in proliferation of squamous NSCLC cell lines. (A) Analysis of PAK1 mRNA expression in laser-capture microdissected lung tissues. Data for Affymetrix probe 226507 at are plotted as the mean (horizontal line), middle 50% of data (box), and 95% confidence interval (lines). Pair-wise comparisons were performed by Student's t-test. Relative to normal tissues (n=9), PAK1 expression was significantly greater in squamous NSCLC (n=16; **, p=0.0005) and adenocarcinoma NSCLC (n=29; p=0.008). The difference in PAK1 mRNA expression between squamous and adenocarcinoma NSCLC was not significant in this panel of lung tumors (p=0.1). (B) Proliferation of a panel of squamous NSCLC cell lines was measured by [³H]thymidine uptake assay. EBC-1, NCI-H520, KNS-62, SK-MES-1 and NCI-H441 cells were transfected with either a non-targeting control siRNA oligonucleotide (black columns) or a pool of siRNA oligonucleotides against PAK1 and PAK2 (white columns). The extent of proliferation under each condition was plotted as a percentage of the normalized non-targeting control value for each cell line and data is shown as the mean±SD.

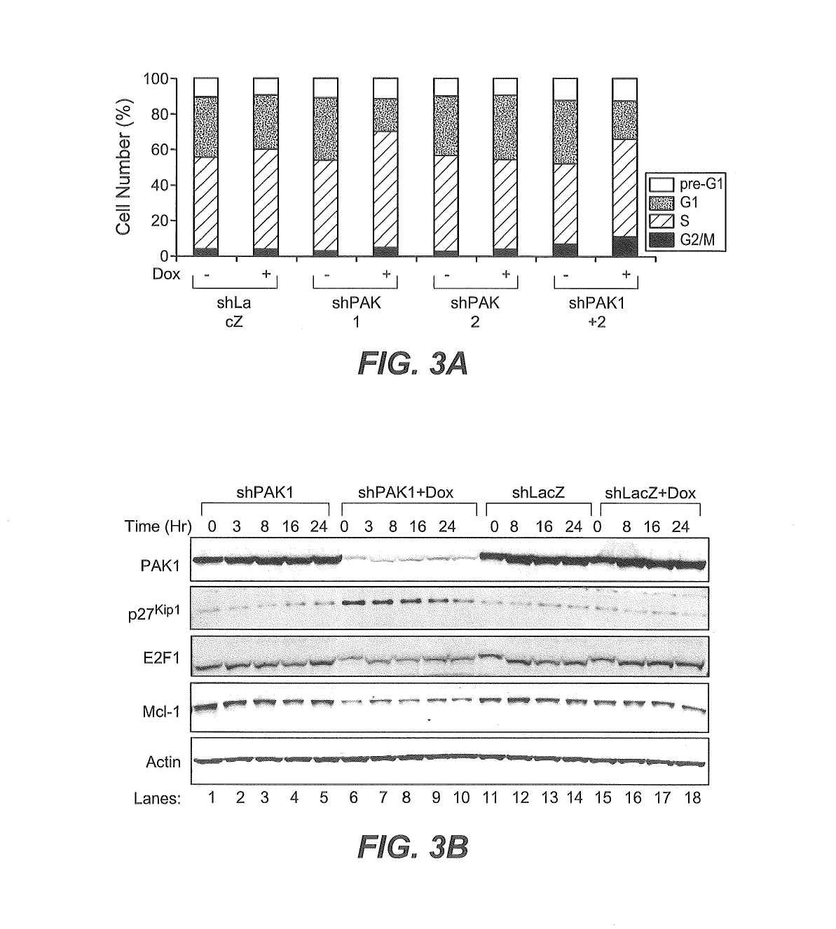

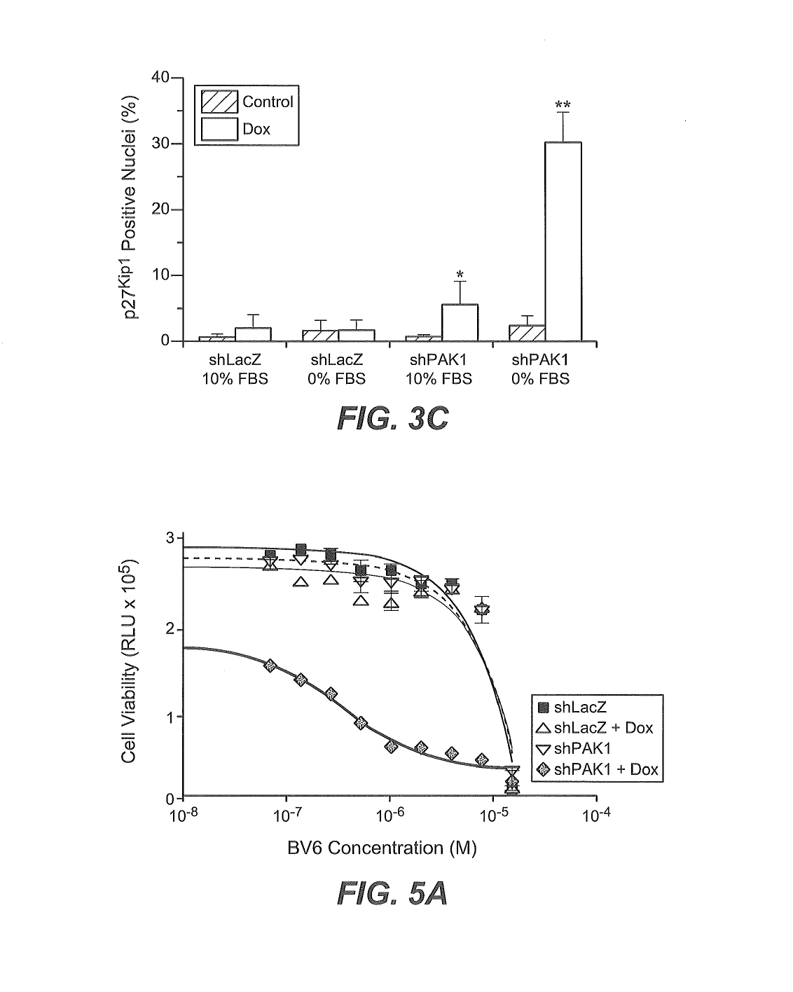

FIG. 3—(A) Accumulation of cells in G₁ phase of the cell cycle is evident following PAK1 knockdown. NCI-H520.X1 cells were treated with 200 ng/mL Dox for 4 days and analyzed by propidium iodide staining and flow cytometry. (B) NCI-H520.X1 cells were serum starved for 24 hours and cell cycle re-entry was monitored by harvesting cell lysates at the indicated time points following growth in 10% serum-containing media. Cell lysates were analyzed by immunoblotting using antibodies against PAK1, p27^{Kip1}, E2F1 and actin. (C) The percentages of cells with nuclear accumulation of p27^{Kip1} are indicated (2000 total cells per condition). Columns represent mean±SD. *, p<0.05. **, p<0.0001.

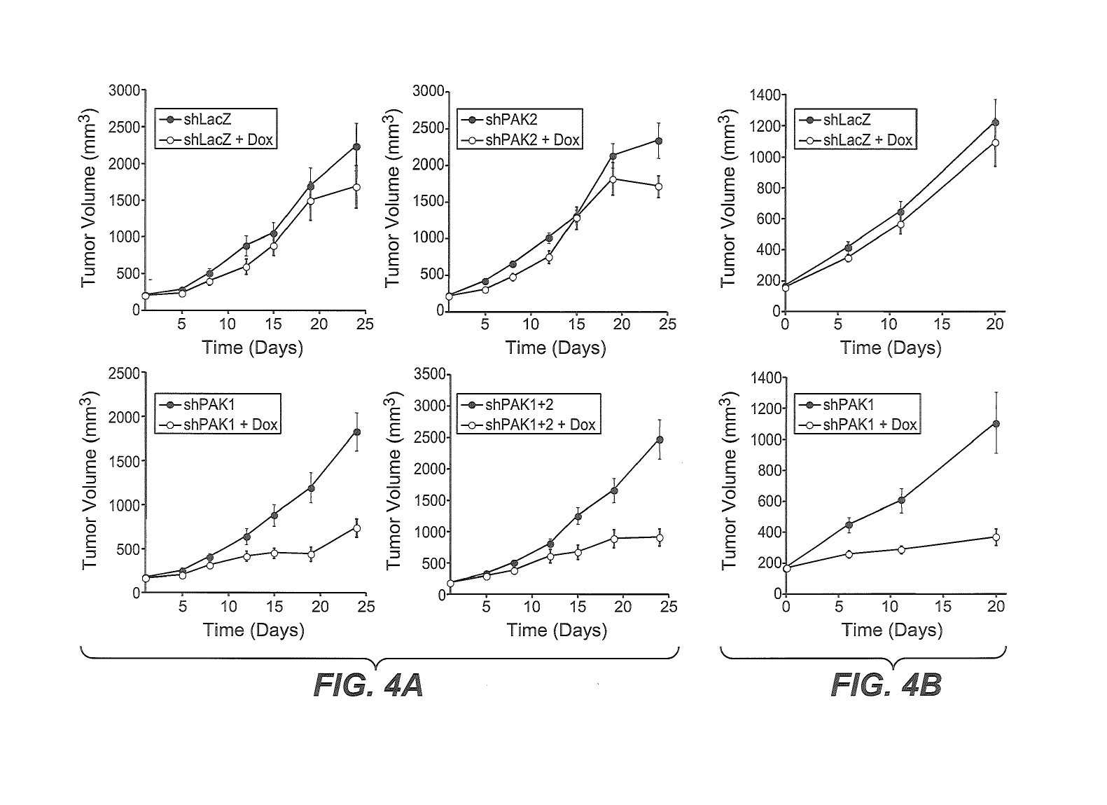

FIG. 4—PAK1 is required for growth of established NCI-H520.X1 and EBC-1 squamous NSCLC tumors. (A) NCI-H520.X1 cells expressing inducible shRNAs against LacZ, PAK1, PAK2 or PAK1+PAK2 were implanted in the flank of athymic mice as described in Materials and Methods. Treatment in each experiment was initiated when tumor size ranged from 200 to 250 mm³. Administration of 1 mg/mL doxycycline via drinking water resulted in inhibition of tumor growth for mice bearing shPAK1 and shPAK1+2 NCI-H520.X1 cells. Induction of PAK2- or LacZ-specific shRNAs did not affect tumor growth kinetics. No animal weight loss was observed. Data consist of 10 mice per treatment group and errors bars represent the standard error. Individual mice were removed from data plotting when tumors reached volume end point of 2000 mm³: shLacZ control n=5; shLacZ+Dox n=3; shPAK1 control n=2; shPAK2 control n=5; shPAK2+Dox n=2; shPAK1+2 control n=5. (B) EBC-1 tumors expressing shLacZ or shPAK1 were allowed to grow to 200-250 mm³ before groups of mice with tumors of equivalent size were administered doxycycline to inhibit PAK1. Data consist of 10 mice per treatment group and errors bars represent the standard error.

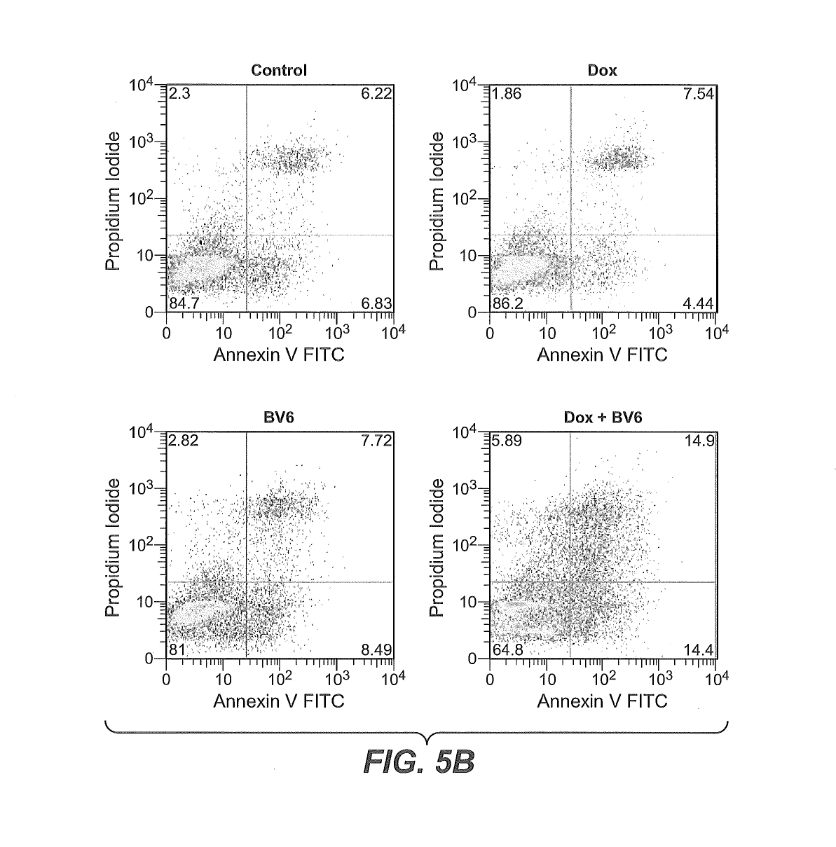

FIG. 5—PAK1 inhibition decreases NF-κB pathway activation and combines with IAP antagonists to promote apoptosis of NSCLC cells. (A) EBC-1-shPAK1 and -shLacZ cells were treated with BV6 IAP antagonist and 300 ng/mL doxycycline (Dox). The highest concentration of BV6 was 20 μM and 2-fold serial dilutions were assessed in a 10-point dilution curve. Cells were pre-incubated in the presence of Dox for 3 days prior to addition of BV6 for an additional 3 days. Cell cultures were then analyzed by a CellTiterGlo viability assay. Data points were performed in quadruplicate. (B) Fluorescence-activated cell sorting analysis for Annexin V and propidium iodide (PI) staining. EBC-1-shPAK1 cells were cultured in the presence or absence of 300 ng/mL doxcycline (Dox) for 72 h and 5 μM BV6 for an additional 24 h. Annexin V/PI staining and fluorescence-activated cell sorting (FACS) analysis was then done to assess apoptosis/necrosis.

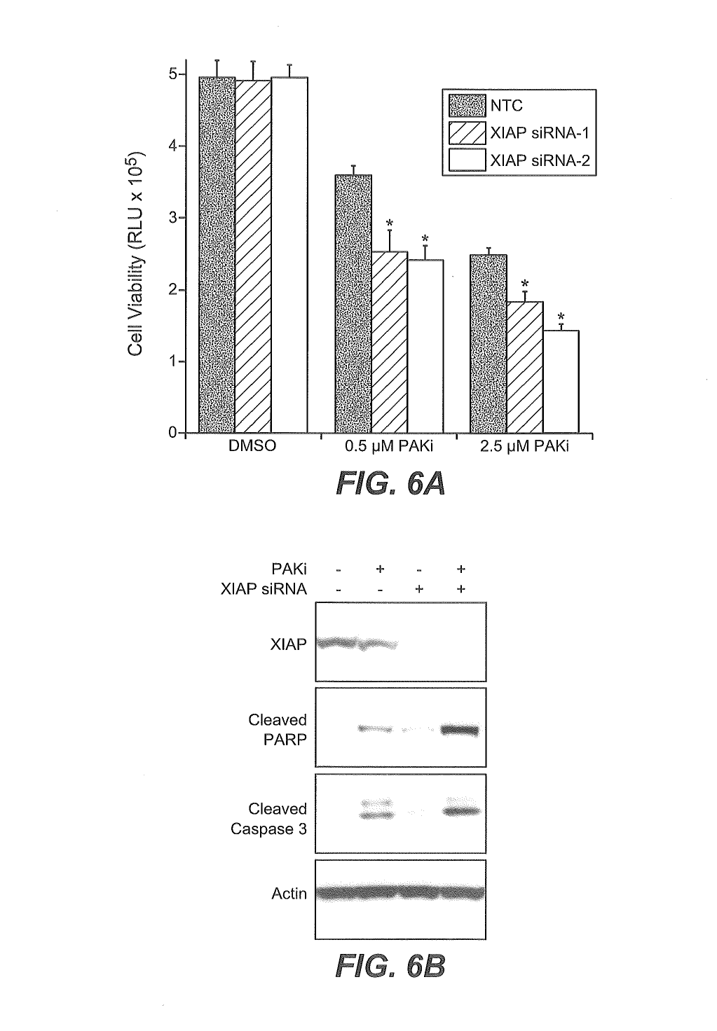

FIG. 6—(A) Down-regulation of XIAP expression potentiates the proapoptotic activity of PF-3758309 PAK small molecule inhibitor (PAK SMI; p<0.0001, Dunnett's t-test). Cells were transfected with non-targeting control (NTC) or XIAP-specific siRNA oligonucleotides for 48 h prior to treatment with DMSO or PAK SMI as indicated for an additional 72 h. Cell viability was determined via Cell Titer Glo assay and results represent mean±standard deviation from three experiments. (B) Combined antagonism of XIAP and PAK1 promotes efficient cleavage of PARP and caspase-3. XIAP siRNA oligonucleotides were transfected for 72 h prior to treatment with DMSO or 5 mM PAK SMI.

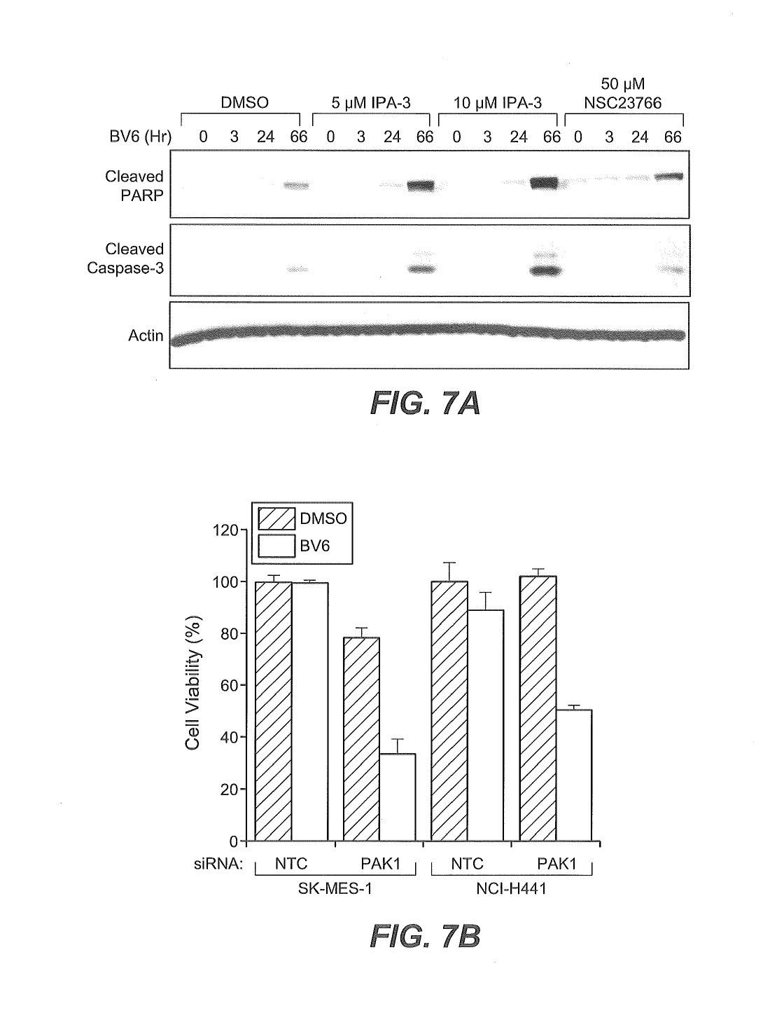

FIG. 7—(A) Combinatorial accumulation of cleaved PARP and caspase-3 by PAK1 and IAP antagonism. Cells were incubated with Dox and 5 μM for indicated time points, lysed and then used for Western blot analysis (B) Dual PAK1 and IAP inhibition results in a synergistic decrease in viability of SK-MES-1 (squamous subtype) and NCI-H441 (adenocarcinoma subtype) NSCLC cells. Cellular ATP consumption was determined via Cell Titer Glo assay following transient siRNA-mediated inhibition of PAK1 and 5 μM BV6 treatment as indicated. Inhibition of cell viability was significantly greater for PAK1 siRNA and BV6 combination than for single agents (p<0.0001, Student's t-test).

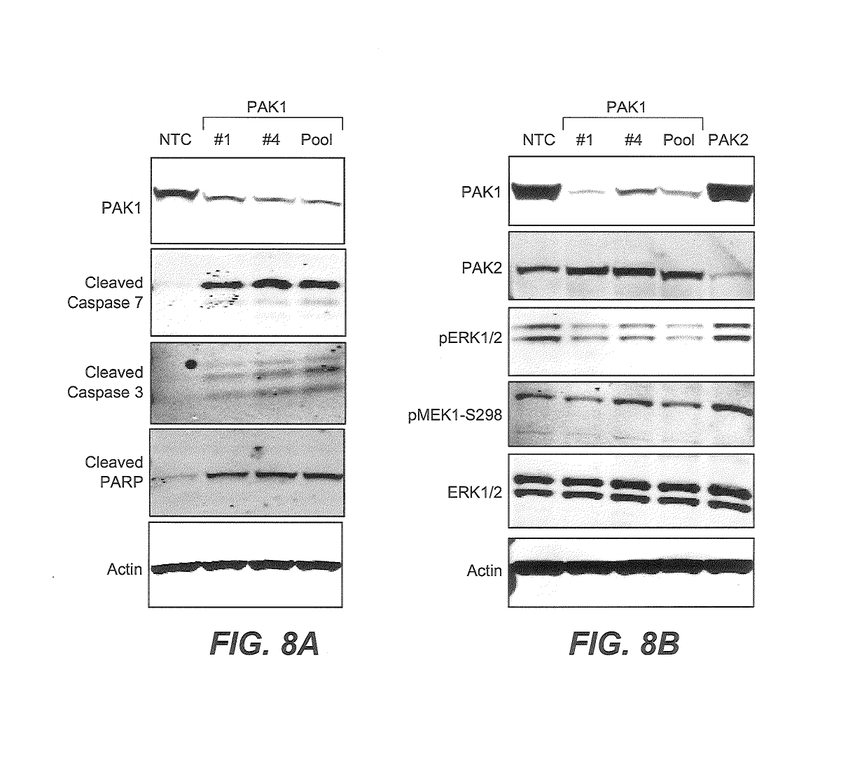

FIG. 8—PAK1 inhibition induces cleavage of caspases and poly ADP ribose polymerase (PARP) in breast cancer cells with focal genomic amplification of PAK1. (A) HCC-1500 cells were transiently transfected with individual or pooled siRNA oligonucleotides (100 nM) to induce PAK1 knockdown. Apoptosis induction was monitored by harvesting cell lysates after 48 h and immunoblotting using antibodies against cleaved caspase-3, cleaved caspase-7 and cleaved PARP. (B) Ablation of PAK1 protein expression in MDA-MB-134 IV cells was also associated with a decrease in MEK1 (Ser298) and ERK1/2 (Thr202/Tyr204) phosphorylation. Total proteins and β-actin were used as controls.

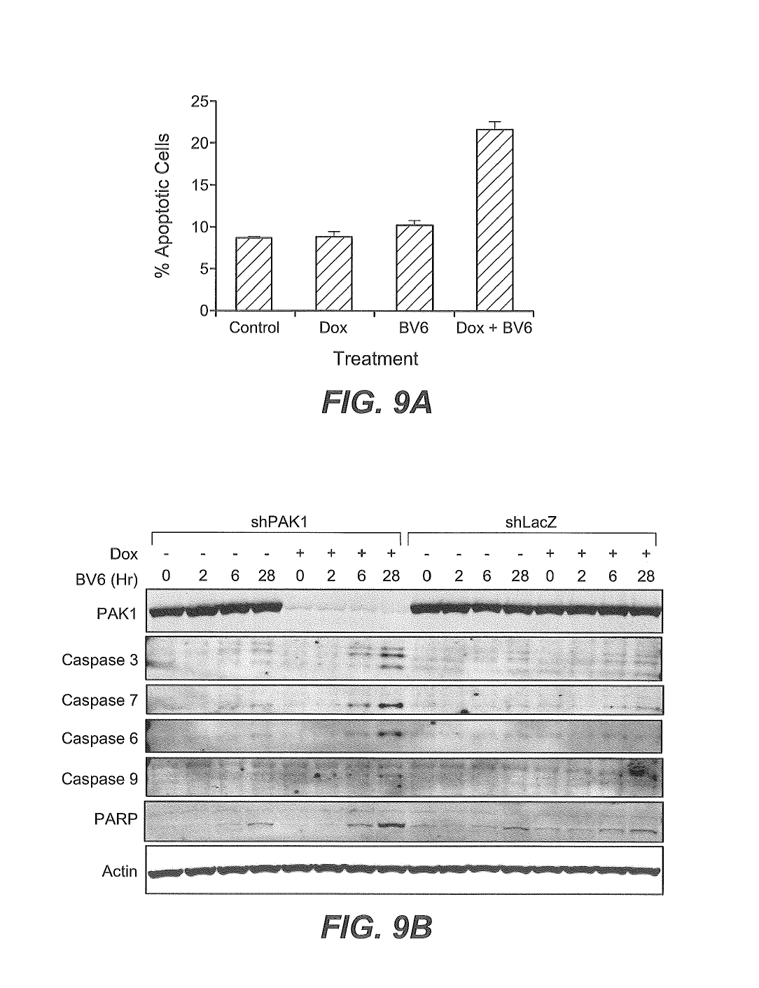

FIG. 9—Combined PAK1 and IAP inhibition results in apoptosis of squamous NSCLC cells. (A) Percentages of Annexin V-positive cells are shown for each treatment condition. (B) Cellular apoptosis markers were increased following genetic ablation of PAK1 and IAP antagonist treatment for indicated times. Cell lysates were analyzed by immunoblotting. PARP cleavage and caspase-3/6/7/9 activation was dramatically elevated by combined Dox and BV6 treatment.

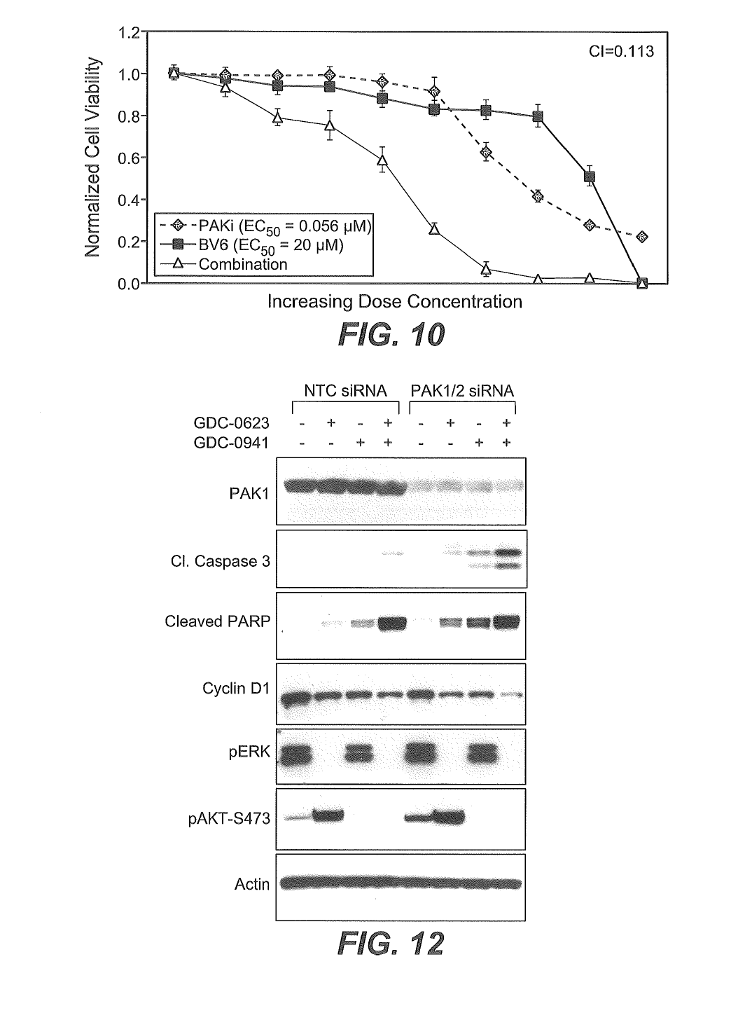

FIG. 10—Combination of ATP-competitive pan-PAK inhibitor PF-3758309 (B. W. Murray et al. Proc. Nat. Acad. Sci. USA 2010 107(20):9446-9471 and IAP small molecule antagonist results in apoptosis of squamous NSCLC cells. (A) Catalytic inhibition of PAK1 via PF-3758309 treatment was tested with BV6 for in vitro combination efficacy in EBC-1 cells using a 4-day CellTiterGlo viability assay. Calcusyn, a program utilizing the Chou and Talalay (Chou T C, Talalay P. Quantitative analysis of dose-effect relationships: the combined effects of multiple drugs or enzyme inhibitors. Adv. Enzyme Regul. 1984; 22:27-55) method of calculating synergy, was used to calculate the combination index and, thus, determine the level of synergy. Strong synergy, as indicated by combination index (CI) values ≦0.3, was observed (CI=0.113). (B) Combination of 5 μM PAK inhibitor (PAKi) and 5 μM BV6 for the indicated times resulted in dramatic induction of cellular apoptotic markers.

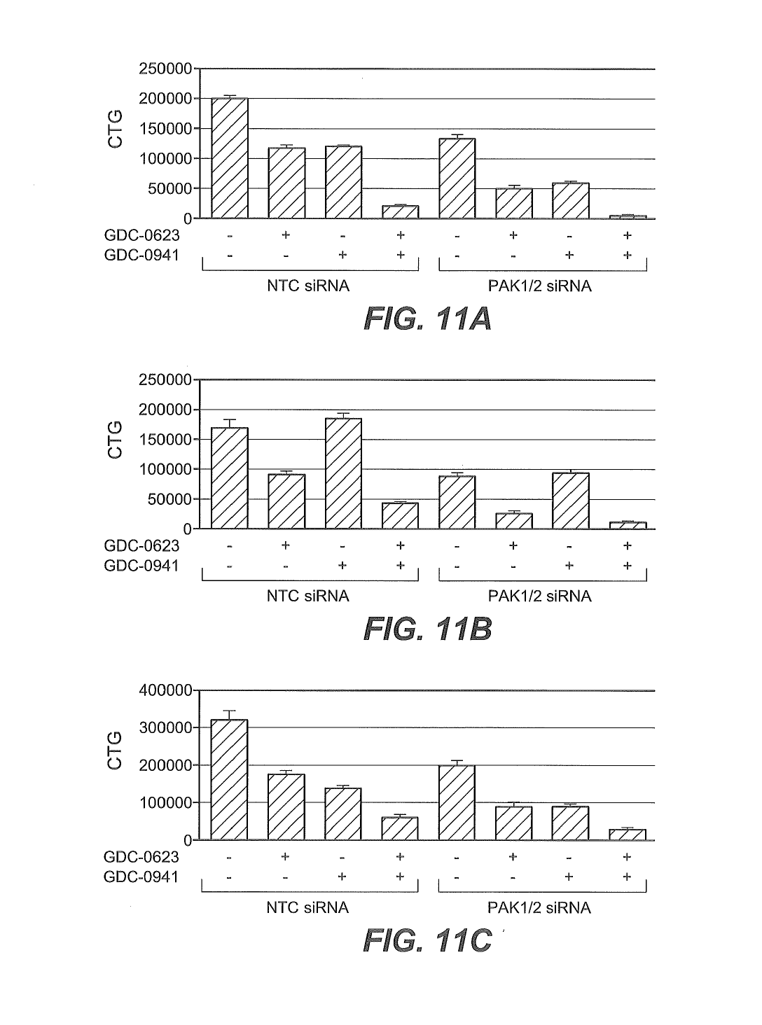

FIG. 11—Combinatorial effect of PAK1, PAK2, MEK and PI3K inhibition on tumor cell viability. CellTiter-Glo® (CTG) assays of cellular viability were performed following PAK1 and PAK2 siRNA transfection and compound treatment for 3 days, as indicated. GDC-0623 is a potent and highly selective inhibitor of MEK1 and MEK2. GDC-0941 is a potent inhibitor of Class I PI3K isoforms with biochemical IC₅₀ values of 3-75 nM for the four Class I isoforms of PI3K. (A) Viability of SKMES-1 (KRAS^{N85K} mutation) lung cancer cells treated with PAK1 and PAK2 siRNA oligonucleotides, 0.2 μM GDC-0623 and 0.5 μM GDC-0941. (B) Viability of Calu-6 (KRAS^{Q61K} mutation) lung cancer cells treated with PAK1 and PAK2 siRNA oligonucleotides, 0.2 μM GDC-0623 and 0.4 μM GDC-0941. (C) Viability of Cal-120 (basal subtype) breast cancer cells treated with PAK1 and PAK2 siRNA oligonucleotides, 2 μM GDC-0623 and 2.5 μM GDC-0941.

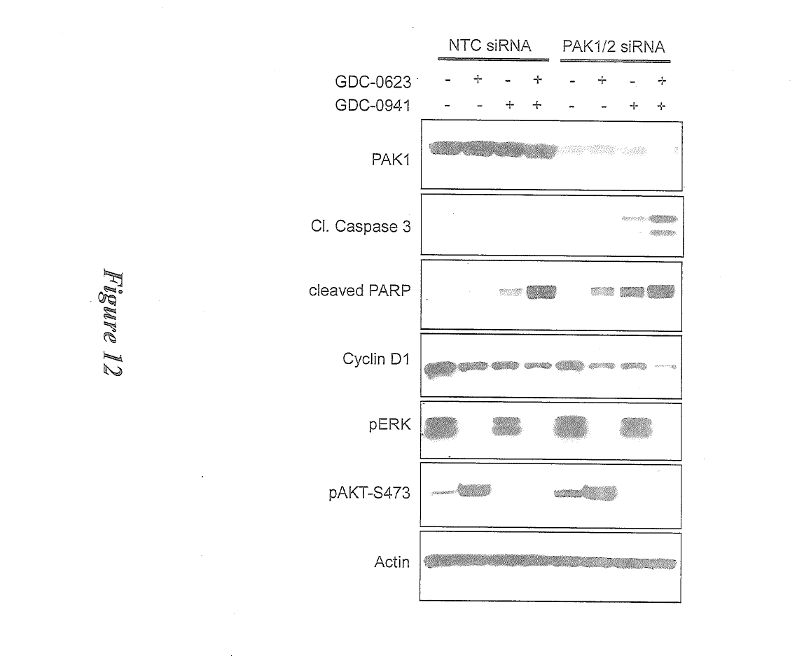

FIG. 12—Combinatorial regulation of apoptotic and proliferation biomarkers following combined PAK1, PAK2, MEK and PI3K inhibition in NSCLC cells. SKMES-1 (KRAS^{N85K} mutant) NSCLC cells were treated with PAK1 and PAK2 siRNA oligonucleotides, 0.4 μM GDC-0623 and 1 μM GDC-0941 for 24 hours. The accumulation of cleaved caspase-3 and poly ADP ribose polymerase (PARD), and decrease of cyclin D1 protein, was enhanced by combination of PAK knockdown with inhibitors of the MEK and PI3K pathways.

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “synergistic” as used herein refers to a therapeutic combination which is more effective than the additive effects of the two or more single agents. A determination of a synergistic interaction between a PAK1 inhibitor and a second anti-hyperproliferative agent may be based on the results obtained from the assays described herein. The combinations provided by this invention have been evaluated in several assay systems, and the data can be analyzed utilizing a standard program for quantifying synergism, additivism, and antagonism among anticancer agents. The program preferably utilized is that described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “carcinoma” refers to an invasive malignant tumor consisting of transformed epithelial cells. The term “squamous cell carcinoma” (SCC) refers to subset of carcinomas that effect squamous epithelial cells that may occur in many different organs, including the skin, lips, mouth, esophagus, urinary bladder, prostate, lungs, vagina, and cervix. It is a malignant tumor of squamous epithelium.

A “chemotherapeutic agent” is a biological (large molecule) or chemical (small molecule) compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, proteins, antibodies, photosensitizers, and kinase inhibitors. Chemotherapeutic agents include compounds used in “targeted therapy” and non-targeted conventional chemotherapy.

Examples of chemotherapeutic agents include erlotinib (TARCEVA®, Genentech/OSI Pharm.), bortezomib (VELCADE®, Millennium Pharm.), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN°, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ11 and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; (ix) anti-angiogenic agents such as bevacizumab (AVASTIN®), Genentech); and (x) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMINITARG®, 2C4, Genentech), trastuzumab (HERCEPTIN®, Genentech) and tositumomab (Bexxar, Corixia.

Humanized monoclonal antibodies with therapeutic potential as chemotherapeutic agents in combination with the PI3K inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, dachzumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

The following abbreviations are used in the present application: DCIS (ductal carcinoma in situ), SCLC (small cell lung cancer), NSCLC (non-small cell lung cancer); SCC (squamous cell carcinoma).

PAKs participate in a number of pathways that are commonly deregulated in human cancer cells. PAK1 is a component of the mitogen-activated protein kinase (MAPK), JUN N-terminal kinase (JNK), steroid hormone receptor, and nuclear factor κβ(NFκβ) signalling pathways, which all have been associated with oncogenesis. PAKs activate MEKand RAF1 by phosphorylating them on serine 298 and serine 338, respectively. The increase of Ras-induced transformation by PAK1 correlated with its effects on signaling through the extracellular signal-regulated kinase (ERK)-MAPK pathway, and was dissociable from effects on the JNK or p38-MAPK pathways. (R. Kumar et al. Nature Rev. Cancer 2006 6:459) Constitutive activation of the ERK/MEK pathway is implicated in the formation; progression and survival of tumors and furthermore is associated with an aggressive phenotype, characterized by uncontrolled proliferation, loss of control of apoptosis and poor prognosis. (J. A. Spicer, Expert Opin. Drug Discov. 2008 3:7)

Tumor formation and progression require the inactivation of pro-apoptotic signals in cancer cells. PAK activity has been shown to downregulate several important pro-apoptotic pathways. PAK1 phosphorylation of RAF1 induces RAF1 translocation to mitochondria, where it phosphoiylates the pro-apoptotic protein BCL2-antagonist of cell death (BAD). PAK1, PAK2, PAK4 and PAK5 have also been reported to directly phosphorylate and inactivate BAD in to selected cell types, such as CV-1 (simian) in origin and carrying the SV40 (COS) kidney, Chinese hamster ovarian (CHO) and human embryonic kidney (HEK) 293T cells. (R. Kumar et al., supra) However, the relevant pathways downstream of PAK1 in human tumor cells remain only partially understood.

PAK1 is widely expressed in a variety of normal tissues; however, expression is significantly increased in ovarian, breast and bladder cancer. (S. Balasenthil et al., J. Biol. Chem. 2004 279:4743; M. Ito et al., J. Urol. 2007 178:1073; P. Schraml et al., Am. J. Pathol. 2003 163:985) In luminal breast cancer, genomic amplification of PAK1 is associated with resistance to tamoxifen therapy, possibly occurring as a result of direct phosphorylation and ligand-independent transactivation of estrogen receptor by PAK1. (S. K. Rayala et al., Cancer Res. 2006. 66:1694-1701) PAK1 is an attractive target for developing therapeutic agents effective for use in treatment of hyperproliferative disorders. (R Kumar et al. supra)

Amplification Experiments—

PAK1 genomic copy number and gene expression were determined for a large panel of breast, lung and head-and-neck tumors. PAK1 genomic amplification was prevalent in luminal breast cancer and PAK1 protein expression was associated with lymph node invasion and metastasis.

Several genomic regions with copy number gains have been identified in breast cancer via comparative genomic hybridization approaches (J. Climent et al., Biochem. Cell Biol. 2007 85:497-508; E.H. van Beers and P. M. Nederlof, Breast Cancer Res. 2006 8:210) An assay of 51 breast tumors for DNA copy number changes using high-resolution single nucleotide polymorphism (SNP) arrays and analyzed data using the Genomic Identification of Significant Targets in Cancer (GISTIC) method (P. M. Haverty et al., Genes Chromosomes Cancer 2008 47:530-542. 21; R. Beroukjim et al., Proc. Natl. Acad. Sci. USA 2007 104:20007-20012). A chromosome 11 region of amplification is shown in FIG. 1A. Two distinct GISTIC peaks were observed at 69 and 76 Mb, suggesting that the 11q13.5 region contains 2 independent amplicons. The 69-Mb peak corresponds to amplification of CCNDI, a very well described genomic alteration in breast cancer. (C. Dickson, et al., Cancer Lett. 1995 90:43-50) The plateau of the 76-Mb peak contains the PAK1 gene (shown as a dotted line). The frequency of PAK1 amplification was 17% (copy number ≧2.5) in this tumor panel and copy number gain was well correlated with mRNA expression (Pearson correlation=0.75; FIG. 1B). Similar results were also obtained in a larger panel (n=165) of breast tumors that were also analyzed for genomic amplification by high-resolution SNP arrays. PAK1 gene amplification was prevalent and mean DNA copy number was greatest in luminal, hormone receptor-positive tumors (7.7 mean copy number) and least in basal breast tumors (2.8 mean copy number). (Z. Kan et al., Nature 2010 466:869-873) These experiments suggest that PAK1 could be a tumor-promoting “driver” gene in the 76-Mb amplicon of chromosome 11. PAK1 expression was absent in normal breast epithelial cells, but was detected in the malignant cells of 39% of primary breast adenocarcinomas.

PAK1 protein expression level and subcellular localization were ascertained via immunohistochemical (IHC) staining of tissue microarrays. Robust and selective IHC reactivity of PAK1 antibody was confirmed in cancer cell lines with immunoblot analysis of protein extracts from these cells performed in parallel. PAK1 protein expression data for 226 primary breast cancers, 15 DCIS, 32 breast cancer lymph node metastases, 97 NSCLC, 27 SCLC and 130 head and neck squamous cell carcinomas are summarized in Table 1. PAK1 staining intensity varied among tumor tissues, ranging from no or low staining to very strong staining in the either the cytoplasm and/or nucleus.

RNA was purified from 88 primary breast cancer specimens and cytoplasmic PAK1 IHC staining was correlated with increased mRNA expression. These data show that PAK1 expression is broadly up-regulated in breast cancer and that high expression is correlated with disease aggressiveness.

Strong nuclear and cytoplasmic PAK1 expression was also prevalent in squamous non-small cell lung and selective PAK1 inhibition was correlated with delayed cell cycle progression in vitro and in vivo.

Expression of PAK1 protein was analyzed on tissue microarrays of 27 SCLCs and 97 NSCLCs, the latter being comprised of 30 adenocarcinomas and 67 SCCs. 43/67 (64%) squamous NSCLC samples were positive for PAK1 expression and 52% of all cases showed staining of moderate (2+) or strong (3+) intensity in the malignant cells. Nuclear localization of PAK1 was also evident in a significant proportion of squamous NSCLC tumors (17/67; 25%). In contrast to squamous cell carcinoma, NSCLC adenocarcinoma (p=0.0008) and SCLC (p=0.003) tumors expressed only weak to moderate levels of PAK1 in the cytoplasm only. Adjacent normal lung tissue did not express appreciable levels of PAK1. Elevated PAK1 expression was also prevalent (79/130; 61%) in head-and-neck tumors, an additional indication of squamous cell carcinoma.

TABLE 1
Combination of PAK1 inhibition with ay small molecule panel

| | Single | Combo | | |
|---|---|---|---|---|
| | agent | PAKᵢ⁸ | Fold | |
| | EC₅₀ | EC₅₀ | change | Mechanism of action and/or approved |
| Compound | (mM) | (mM) | in EC₅₀ | use |
| BV6 | 20 | 0.35 | 57.54 | Antagonist of inhibitor of apoptosis |
| | | | | (IAP) proteins |
| erlotinib¹ | 20 | 1.56 | 15.02 | Epidermal growth factor receptor |
| | | | | (EGFR) inhibitor for NSCLC |
| gefitinib² | 20 | 3.3 | 12.8 | EGFR kinase inhibitor |
| G-416 | 20 | 1.67 | 12 | Antagonist of IAP proteins |
| U0126 | 19.46 | 2.3 | 8.45 | Inhibitor of MAPK/ERK kinase-1/2 |
| | | | | (MEK1/2) |
| lapatinib³ | 6.31 | 0.86 | 7.34 | EGFR/HER2 inhibitor used for HER2- |
| | | | | positive breast cancer |
| dasatinib⁴ | 8.29 | 1.53 | 5.43 | Dual BCR/ABL and Src family kinase |
| | | | | inhibitor for CML |
| altretamine⁵ | 20 | 3.94 | 5.07 | Alkylating chemotherapy used for |
| | | | | refractory ovarian cancer |
| oxaliplatin⁶ | 10.5 | 2.48 | 4.23 | Platinum-based chemotherapy used for |
| | | | | colorectal cancer |
| ZD6474⁷ | 20 | 6.88 | 2.91 | VEGF and EGF inhibitor in clinical |
| | | | | development |
| Akt inhibitor VIII | 3.21 | 2.03 | 1.58 | Akt-½ kinase inhibitor |

Trade Names:
¹Tarceva,
²Iressa,
³Tykerb,
⁴Sprycel,
⁵Hexalen,
⁶Eloxatin,
⁷Vandetanib
⁸PAK1 knockdown shRNA and compound.

In head and neck tumors elevated PAK1 expression was also prevalent (79/130; 61%) in head-and-neck tumors, another indication of squamous cell carcinoma (Table 1).

Anti-tumor efficacy of PAK1 inhibition in preclinical tumor models of squamous NSCLC-Inhibition of PAK1 expression using shRNA in NCI-H520.X1 and EBC-1 xenograft models resulted significant inhibition of tumor growth (FIGS. 4A and B). Following tumor establishment (200-250 mm³), animals were administered Dox in sucrose drinking water and tumor growth was monitored for 21-24 days. For NCI-H520.X1 tumor-bearing animals, inhibition of PAK1, but not PAK2, significantly impaired tumor growth relative to control shLacZ mice as measured on the final day of dosing (Dunnett's t-test, p<0.0001; FIG. 4A). Combined knockdown of PAK1 and PAK2 resulted in inhibition of tumor growth that was comparable to that of PAK1 inhibition alone (63.7% and 59.7%, respectively). Equivalent results were obtained using EBC-1 tumor xenografts and 66.8% inhibition of tumor growth was observed following in vivo knockdown of PAK1 (FIG. 4B). Lastly, tumor progression data for both xenograft models were analyzed for the time required for tumor size to double from the onset of treatment. By this metric, PAK1 inhibition also resulted in a significant anti-tumor effect compared to the other cohorts (p<0.0001). Dox treatment was well tolerated and no animals exhibited any appreciable body weight loss. Analysis of xenograft tumors by immunohistochemistry revealed a substantial decrease in Ki-67 positive tumor cells in Dox-treated tumors expressing shPAK1 compared to shLacZ controls. The proportion of Ki-67 positive nuclei was quantified and the anti-proliferative effect of PAK1 knockdown in vivo was shown to be statistically significant (p<0.01; NCI-H520.X1 shLacZ 69±6%; NCI-H520.X1 shPAK1 50±4%; EBC-1 shLacZ 91±3%; EBC-1 shPAK1 75±11%). PAK1 and PAK2 levels were reduced by greater than 80% in Dox-treated tumors and PAK1 knockdown was not associated with decreased AKT activation as has been suggested. (T.C. Hallstrom and J.R. Nevins, Cell Cycle 2009 8:532-535.)

Analysis of cell lines with PAK1 genomic copy number gain revealed a dependence on PAK1 expression and activity for cell survival Inhibition of PAK1 catalytic activity using IPA-3, an allosteric inhibitor that prevents PAK1-3 activation by Rho family GTPases (J. Viaud, J. and J.R. Peterson, Mol. Cancer. Ther. 2009 8:2559-2565), resulted in a pronounced induction of apoptosis as determined by fluorescence-activated cell sorting analysis for Annexin-V/propidium iodide staining of dying cells (7-fold increase; FIG. 1D). This phenotype was evident within 24-48 h and was confirmed using selective, siRNA-mediated knockdown of PAK1 expression (2-6-fold increase in Annexin-V incorporation; FIG. 1C). Cell death induced by PAK1 inhibition was also associated with caspase activation, PARP cleavage and attenuated phosphorylation of MEK1-S298 and ERK1/2 (FIG. 8). Hence, the strong induction of cell death resulting from PAK1 inhibition in breast cancer cells with PAK1 amplification suggests that this kinase contributes to the oncogenic phenotype, at least in part, by suppressing tumor cell apoptosis.

The dependence on PAK1 suggests it may be an “Achilles' heel” for a subpopulation of breast cancer provides evidence of oncogene addiction (I.B. Weinstein and A. Joe, Cancer Res. 2008 68:3077-3080) and a rationale for PAK1-directed therapy in this disease indication.

The aberrant cytoplasmic expression of PAK1 in greater than 50% of squamous non-small cell lung cancers and in head and neck squamous cell carcinoma further suggest they also may be dependent on PAM expression for continued growth and survival.

At present additional known genetic aberrations in squamous NSCLC include p53, p16^{Ink4a}, PTEN and LKB1 loss-of-function via mutation or methylation, and activating mutations or amplification of protein kinases, such as EGFR, MET, HER2 and PIK3CA. (R. S. Herbst et al., N. Engl. J. Med. 2008 359:1367-1380) Thus inhibition of PAK1 enzymatic activity or scaffold function might combine synergistically with therapeutic agents that target these critical growth and survival pathways to increase anti-tumor efficacy and tumor cell death in tumor cells that over-amplify or over-express PAK1. Such tumor cells include, but are not limited to DCIS, squamous NSCLC and head and neck SCC.

Inhibitors of PAK kinases have been described. (D. Bouzida et al WO2006072831 published Jul. 13, 2006; C. Guo et al., WO2007023382 published Mar. 1, 2007, L. Dong et al., WO2007072153 published Jun. 30, 2007; D. Campbell et al., WO2010/071846 published Jun. 24, 2010; K. Daly et al., US20090275570 published Nov. 5, 2009)

Combination of shRNA induced PAK1 knockdown with molecularly targeted therapeutics induces apoptosis of NSCLC cells—Small molecule library was screened to identify potent synergistic interactions between PAK1 antagonists and other anti-hyper-proliferative agents (Table 1).

A cellular viability screen was performed using EBC-1-shPAK1 isogenic cells and a panel of 200 small molecule compounds that included Food and Drug Administration (FDA) approved oncology drugs, signaling pathway inhibitors and DNA damaging agents. Among the tested compounds, antagonists of inhibitor of apoptosis proteins (IAP; 12 and 57-fold), epidermal growth factor receptor (EGFR; 2.9, 7.4, 12.8 and 15-fold), MAPK/ERK kinase-1/2 (MEK1/2; 8.5-fold) and Src family kinases (5.4-fold) displayed dramatically enhanced efficacy in combination with PF-3758309 (Table 1). None of these agents demonstrated a profound single agent effect (EC₅₀, >6 μM) on the growth and survival of EBC-1 cells in the absence of PAK1 inhibition. Thus, PAK1 inhibition can greatly augment the efficacy of several classes of well-characterized molecularly targeted therapeutics.

Inhibitors of MEK kinase (S. Price, Expert Opin. Ther. Patents 2008 18(6):603-626; E. M. Wallace et al., Curr. Topics Med. Chem. 2005 5(2):215), Akt (C. Lindsley, Curr. Top. Med. Chem. 2010 10:458-477; S.E. Ghayad and P.A. Cohen, Rec. Pat. Anti-Cancer Drug Discov. 2010 5:29-57), Sic (X. Cao et al., Mini-Rev. Med. Chem. 2008 8:1053-1063) and Inibitor of Apoptosis Proteins (TAP) (D. Vucic and W.I. Fairbrother, Clin. Cancer Res, 2007 13(20)5995; A. D. Schimmer and S. Dalili, Hematology 2005 215) have been reviewed.

The prosurvival activity of IAP proteins is antagonized by the second mitochondrial activator of caspases (SMAC) (C. Du et al., Cell 2000 102:33-42; A. M. Verhagen, et al. Cell 2000 102:43-53) and a number of antagonists have been described that mimic SMAC amino-terminal peptides to disrupt the association of IAP with SMAC and activated caspase-9 (K. Zobel et al., ACS Chem. Biol. 2006 1:525-533; E. Varfolomeev et al., Cell 2007 131:669-681). In particular, BV6 (C. Ndubaku et al., Future Med. Chem. 2009 1(8):1509) represents one such class of small molecule antagonist that binds to baculovirus TAP repeat (BIR) domains and promotes rapid auto-ubiquitination and proteasomal degradation of c-TAP 1 and cIAP-2 (Zobel supra). Consistent with the small molecule screening data, strong combinatorial activity was confirmed for dual inhibition of PAK1 and IAP in EBC-1 cells (FIG. 5A). This dramatic increase in BV6 potency on EBC-1-shPAK1 cells when co-treated with Dox (EC₅₀=4.1×10⁻⁷ μM) relative to controls (EC₅₀=3.0×10⁻³ μm) translated into a strong induction of cellular apoptosis as determined by Annexin-V flow cytometry assay (FIG. 5B) and immunoblotting for cleaved caspases-3, 6, 7 and 9 (FIG. 9). Importantly, evidence of enhanced cell killing was also observed using pharmacological inhibitors of PAK (IPA-3, PF-3758309) or Racl (NSC23766) catalytic activity (FIG. 7A). To further explore this apparent synergy we investigated possible molecular mechanisms of PAK1 and IAP inhibition on the induction of apoptosis. Combined PAK1 and TAP inhibition did not involve either altered kinetics of IAP protein degradation induced by BV6 or increased autocrine signaling by TNFα. Lastly, additional NSCLC cell lines, including those that are only minimally responsive to the activity of either single agent, were examined for anti-tumor efficacy resulting from combined inhibition of PAK1 and IAP. Transient siRNA-mediated knockdown of PAK1 expression followed by BV6 treatment resulted in a significant reduction of SK-MES-1 and NCI-H441 cell viability (FIG. 7B).

Combined inhibition of PAK1 and PAK2 with inhibitors of MEK (GDC-0623) and PI3K (GDC-0941; A. J. Folkes et al., J. Med. Chem. 2008 57:5522-5532) pathways was also examined. Combinatorial efficacy, as determined by reduced cellular viability (FIG. 11) and induction of apoptotic biomarkers (FIG. 12), was observed following inhibition of PAK, MEK and PI3K signaling. Taken together, these studies provide strong preclinical support that breast and NSCLC may provide several opportunities for rational combination therapies with PAK1 inhibitors. In one embodiment of the present invention there is provided a method of treating tumors comprising contacting the tumor with a PAK 1 inhibitor and a second anti-hyperproliferative compound.

In another embodiment of the present invention there is provided a method of treating tumors comprising contacting the tumor with a PAK 1 inhibitor and an inhibitor or antagonist of EGFR, the Raf/MEK/ERK pathway, Src, PI3K/AKT/mTOR pathway or inhibitor of apoptosis proteins.

In another embodiment of the present invention there is provided a method for treating a tumor wherein said tumor exhibits elevated levels of a PAK1 comprising contacting the tumor with a PAK 1 inhibitor and a second anti-hyperproliferative compound.

In another embodiment of the present invention there is provided a method for treating a tumor wherein said tumor exhibits elevated levels of PAK1 comprising contacting the tumor with a PAK 1 inhibitor and an inhibitor or antagonist of EGFR, the Raf/MEK/ERK pathway, Src, PI3K/AKT/mTOR pathway or inhibitor of apoptosis proteins.

In one embodiment of the present invention there is provided a method of treating tumors wherein said tumor is breast tumor, a squamous non-small cell lung tumor or a squamous head and neck tumor comprising contacting the tumor with a PAK 1 inhibitor and a second anti-hyperproliferative compound.

In another embodiment of the present invention there is provided a method of treating tumors wherein said tumor is breast tumor, a squamous non-small cell lung tumor or a squamous head and neck tumor comprising contacting the tumor with a PAK 1 inhibitor and an inhibitor or antagonist of EGFR, the Raf/MEK/ERK pathway, Src, Akt or a inhibitor of apoptosis proteins.

In another embodiment of the present invention there is provided a method for treating a tumor wherein said tumor is breast tumor, a squamous non-small cell lung tumor or a squamous head and neck tumor that exhibits elevated levels of a PAK1 comprising contacting the tumor with a PAK 1 inhibitor and a second anti-hyperproliferative compound.

In another embodiment of the present invention there is provided a method for treating a tumor wherein said tumor is breast tumor, a squamous non-small cell lung tumor or a squamous head and neck tumor that exhibits elevated levels of a PAK1 comprising contacting the tumor with a PAK 1 inhibitor and an inhibitor or antagonist of EGFR, the Raf/MEK/ERK pathway, Src, Akt or inhibitor of apoptosis proteins.

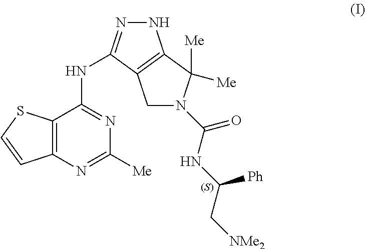

In one embodiment of the present invention there is provided a method of treating tumors comprising contacting the tumor with the compound of formula I (PF-3758309) and a second anti-hyperproliferative compound.

In another embodiment of the present invention there is provided a method of treating tumors comprising contacting the tumor with the compound of formula I and an inhibitor or antagonist of EGFR, the Raf/MEK/ERK pathway, Src, Akt or inhibitor of apoptosis proteins.

In another embodiment of the present invention there is provided a method for treating a tumor wherein said tumor exhibits elevated levels of a PAK1 comprising contacting the tumor with the compound of formula I and a second anti-hyperproliferative compound.

In another embodiment of the present invention there is provided a method for treating a tumor wherein said tumor exhibits elevated levels of PAK1 comprising contacting the tumor with the compound of formula I and an inhibitor or antagonist of EGFR, the Raf/MEK/ERK pathway, Src, Akt or inhibitor of apoptosis proteins.

In another embodiment of the present invention there is provided a method for treating a tumor wherein said tumor is breast tumor, a squamous non-small cell lung tumor or a squamous head and neck tumor that exhibits elevated levels of a PAK1 comprising contacting the tumor with the compound of formula I and a second anti-hyperproliferative compound.

In another embodiment of the present invention there is provided a method for treating a tumor wherein said tumor is breast tumor, a squamous non-small cell lung tumor or a squamous head and neck tumor that exhibits elevated levels of a PAK1 comprising contacting the tumor with the compound of formula I and an inhibitor or antagonist of EGFR, the Raf/MEK/ERK pathway, Src, Akt or inhibitor of apoptosis proteins.

In one embodiment of the present invention there is provided a method of treating tumors comprising contacting the tumor with the compound of formula I and an inhibitor of inhibitor of apoptosis proteins.

In another embodiment of the present invention there is provided a method for treating a tumor wherein said tumor exhibits elevated levels of a PAK1 comprising contacting the tumor with the compound of formula I and an inhibitor of inhibitor of apoptosis proteins.

In one embodiment of the present invention there is provided a method of treating tumors comprising contacting the tumor with the compound of formula I and BV6 or G24416.

In another embodiment of the present invention there is provided a method for treating a tumor wherein said tumor exhibits elevated levels of a PAK1 comprising contacting the tumor with the compound of formula I and BV6 or G24416.

In one embodiment of the present invention there is provided a method of treating tumors comprising contacting the tumor with the compound of formula I and an EGFR inhibitor antagonist.

In another embodiment of the present invention there is provided a method for treating a tumor wherein said tumor exhibits elevated levels of a PAK1 comprising contacting the tumor with the compound of formula I and an EGFR inhibitor or antagonist.

In one embodiment of the present invention there is provided a method of treating tumors comprising contacting the tumor with the compound of formula I and erlotinib, gefitinib or lapatinib.

In another embodiment of the present invention there is provided a method for treating a tumor wherein said tumor exhibits elevated levels of a PAK1 comprising contacting the tumor with the compound of formula I and erlotinib, gefitinib or lapatinib.

In one embodiment of the present invention there is provided a method of treating tumors comprising contacting the tumor with the compound of formula I and an inhibitor of the Ras/Raf/MEK/Erk signaling cascade.

In another embodiment of the present invention there is provided a method for treating a tumor wherein said tumor exhibits elevated levels of a PAK1 comprising contacting the tumor with the compound of formula it and an inhibitor of the Ras/Raf/MEK/Erk signaling cascade.

In one embodiment of the present invention there is provided a method of treating tumors comprising contacting the tumor with the compound of formula I and an inhibitor of Akt kinase.

In another embodiment of the present invention there is provided a method for treating a tumor wherein said tumor exhibits elevated levels of a PAK1 comprising contacting the tumor with the compound of formula I and an inhibitor of Akt kinase.

In one embodiment of the present invention there is provided a method of treating tumors comprising contacting the tumor with the compound of formula I and an inhibitor of Src kinase.

In another embodiment of the present invention there is provided a method for treating a tumor wherein said tumor exhibits elevated levels of a PAK1 comprising contacting the tumor with the compound of formula I and an inhibitor of Src kinase.

In another embodiment of the present invention there is provided a method of treating a patient suffering from a cancer or a hyperproliferative disorder comprising co-administering to a patient in need thereof a PAK 1 inhibitor and a second anti-hyperproliferative agent.

In another embodiment of the present invention there is provided a method of treating a patient suffering from a cancer or a hyperproliferative disorder comprising co-administering to a patient in need thereof a PAK 1 inhibitor and an inhibitor or antagonist of EGFR, the Raf/MEK/ERK pathway, Src, Akt or inhibitor of apoptosis proteins.

In another embodiment, the present invention provides a combination of a PAK1 inhibitor with a second anti-hyperproliferative compound for the treatment of tumors.

In another embodiment, the present invention provides a co-administration of a PAK 1 inhibitor and a second anti-hyperproliferative agent for the treatment of a cancer or a hyperproliferative disorder.

In another embodiment, the present invention provides the use of a combination of a PAK1 inhibitor with a second anti-hyperproliferative compound for the preparation of a medicament for the treatment of tumors.

In another embodiment, the present invention provides the use of a PAK 1 inhibitor and a second anti-hyperproliferative agent for the preparation of a medicament for the treatment of a cancer or a hyperproliferative disorder.

The following examples illustrate the biological evaluation of compounds within the scope of the invention. These examples which follow are provided to enable those skilled in the art to more clearly understand and to practice the present invention. They should not be considered as limiting the scope of the invention, but merely as being illustrative and representative thereof.

### EXAMPLE 1

### Tissue Samples

Formalin-fixed paraffin-embedded tissue blocks and corresponding pathology reports were obtained for 97 sequential NSCLC and 27 sequential SCLC, 130 head and neck SCC, 15 DCIS, 226 primary breast cancers and 32 breast cancer lymph node metastases (John Radcliffe Hospital, Oxford, UK). Tissue microarrays (TMAs) were assembled as described previously (L. Bubendorf, et al., J. Pathol. 2001 195:72-79).

For the sequential patients with breast adenocarcinoma, surgery was performed between 1989 and 1998, and patients were treated with a wide local excision and postoperative radiotherapy or mastectomy with or without postoperative radiotherapy. Patients received adjuvant chemotherapy and/or adjuvant hormone therapy, or no adjuvant treatment. Tamoxifen was used as endocrine therapy for 5 years in estrogen receptor (ER) positive patients. Patients who were <50 years of age, with lymph node positive tumors, or ER- and/or a primary tumor >3 cm in diameter, received adjuvant cyclophosphamide, methotrexate, and 5-fluorouracil (CMF) for six cycles, in a three weekly intravenous regimen. Patients ≧50 years of age with ER-, lymph node-positive tumors also received CMF. Estrogen receptor (ER) content was determined using an enzyme-linked immunosorbent assay technique (Abbott Laboratories, Abbott Park, Ill.). Tumors were considered positive when cytosolic ER levels were >10 fmol/mg of total cytosolic protein. HER2 status was assessed with the HercepTest (DAKO, Carpinteria, Calif.). Receptor values were monitored by participation in the EORTC quality control scheme.

The operable NSCLC series comprised surgical resection specimens from 30 adenocarcinomas and 67 squamous cell carcinomas (surgery was performed from 1984 to 2000). Clinical and pathological data were available for 75 cancers. Thirty-five cases (47%) were stage T1 and 40 cases (53%) were stage T2. Fifty-three cases (71%) were stage NO and twenty-two cases (29%) were stage N1. Patients did not receive adjuvant chemotherapy and information regarding radiotherapy was not available.

The head and neck squamous cell carcinoma series comprised surgical resection specimens from 11 oropharyngeal cancers, 27 cancers arising in the oral cavity, 17 laryngeal cancers and 75 hypopharyngeal cancers (definitive surgery was performed from 1995 to 2005). Nine cancers were UICC stage 1, 16 were stage 2, 29 were stage 3 and 76 were stage 4. Post-operatively 108 patients (83%) received radiotherapy.

### EXAMPLE 2

### Genome Copy Number and Expression Analysis

For the Affymetrix 500K SNP array analysis, genomic DNA preparation, chip processing and data analysis were performed as published previously. (P. M. Haverty et al., Genes Chromosomes Cancer 2008 47:530-542) Regions of significant gains or losses were identified using the GISTIC (Genomic Identification of Significant Targets in Cancer) algorithm (R. Beroukhim, et al., Proc Natl Acad Sci USA 2007 104:20007-20012). To collect expression array data for matched tumor samples, RNA was extracted from frozen tissue from 88 cases of the primary breast cancer series and applied to Affymetrix (Santa Clara, Calif.) HGU133 gene expression microarrays. Gene probe intensity data were used to subclassify the tumors into basal, luminal-A, luminal-B, Her2 and normal types according to published criteria (C. M. Perou et al., 2000 Nature 2000 406:747-752). The 226507 at probeset was chosen to represent PAK1 mRNA expression.

### EXAMPLE 3

### Cell Culture and Viability Assays

Cell lines were acquired from either the Health Science Research Resources Bank (HSRRB, Japan) or American Type Culture Collection (ATCC; Manassas, Va.) and maintained at 37° C. and 5% CO₂ in Dulbecco's Modified Eagle Medium (DMEM) or Roswell Park Memorial Institute 1640 (RPMI 1640) media with 10% fetal bovine serum and 4 mM L-glutamine. For analysis of cell proliferation by thymidine incorporation into DNA, cells were incubated with 1 μCi/well [³H]thymidine for 18 h and harvested onto Unifilter® GF/C plates using a Filtermate™ 196 harvester (Perkin Elmer, Waltham, Mass.). MicroScint™ 20 liquid scintillation cocktail was added to the dried filter plates that were subsequently sealed and counted in a Topcount™ (Perkin Elmer, Waltham, Mass.). For cell cycle analysis via flow cytometry, cells at a density of 1×10⁶ were fixed in 70% ice-cold ethanol for 1 hour and then washed with PBS and incubated in propidium iodide (PI) solution (0.05 mg/ml RNase solution (Sigma, St. Louis, Mo.), 0.05 mg/ml Pf (Sigma, St. Louis, Mo.), in PBS) for 3 hours at 4° C. Cells were immediately analyzed with a FacScan flow cytometer (Becton Dickinson, San Jose, Calif.).

To ascertain the role of PAK1 in cell survival, the quantity of cytoplasmic histone-associated DNA fragments was quantified using the Cell Death Detection ELISA Plus kit from Roche (Mannheim, Germany). Alternatively, for cell death analyses via flow cytometry, cells were collected by centrifugation and stained with Annexin V-FITC and PI solution (BD Biosciences, San Jose) according to the manufacturer's instructions.

For the pharmarray viability screen using a 200 compound library, EBC1-shPAK1 cells were cultured in complete growth medium and either untreated or treated with 300 ng/mL doxycycline for 3 days prior to compound addition. Cells were then replated at appropriate density in 384-well plates and treated with 6 concentrations (4-fold serial dilutions from 10 μM) of each compound for 72 hr treatment. Cell viability was assessed via ATP content using the CellTiter-Glo® Luminescent Assay (Promega, Madison, Wis.). Cell growth inhibition and EC₅₀ differences were determined for PAK1 knockdown and wild-type cells.

### EXAMPLE 4

### RNA Interference and Generation of Inducible-shRNA Cell Pools

Short interfering RNA (siRNA) oligonucleotides for PAK1 and PAK2 were obtained from Dharmacon RNAi Technologies (Chicago, Ill.). Short-hairpin RNA oligonucleotides used in this study are as follows: LacZ shRNA (sense) 5′-CTT ATA AGT TCC CTA TCA GTG ATA GAG ATC CCC AAT AAG CGT TGG CAA TTT ATT CAA GAG ATA AAT TGC CAA CGC TTA TTT TTT TTG GAA-3′, LacZ shRNA (antisense) 5′-TTC CAA AAA AAA TAA GCG TTG GCA ATT TAT CTC TTG AAT AAA TTG CCA ACG CTT ATT GGG GAT CTC TAT CAC TGA TAG GGA ACT TAT AAG-3′, PAK1 shRNA-1 (sense) 5′-GAT CCC CGA AGA GAG GTT CAG CTA AAT TCA AGA GAT TTA GCT GAA CCT CTC TTC TTT TTT GGA AA-3′, PAK1 shRNA-1 (antisense) 5′-AGC TTT TCC AAA AAA GAA GAG AGG TTC AGC TAA ATC TCT TGA ATT TAG CTG AAC CTC TCT TCG GG-3′, PAK2 shRNA-3 (sense) 5′-GAT CCC CCT GCA TAA CCT GAA TGA AAT TCA AGA GAT TTC ATT CAG GTT ATG CAG TTT TTT GGA AA-3′, PAK2 shRNA-3 (antisense) 5′-AGC TTT TCC AAA AAA CTG CAT AAC CTG AAT GAA ATC TCT TGA ATT TCA TTC AGG TTA TGC AGG GG-3′. Inducible-shRNA bearing lentivirus constructs were made based on previously described methods (K. P. Hoeflich et al., Cancer Res. 2006 66:999-1006; J. Climent et al., Biochem. Cell Biol. 2007 85:497-508.) by co-transfecting pHUSH-Lenti-GFP and/or pHUSH-Lenti-dsRed constructs containing a desired shRNA with plasmids expressing the vesicular stomatitis virus (VSV-G) envelope glycoprotein and HIV-1 packaging proteins (GAG-POL) in HEK293T cells using Lipofectamine™ (Invitrogen, Carlsbad, Calif.). Target cells were transduced with these viruses and sterile sorted (top 2-5%) by flow cytometry for presence of dsRed or GFP or both. Cells were characterized for doxycycline-inducible protein knockdown by western blot analysis.

### EXAMPLE 5

### Immunoblotting and Immunofluorescence

Frozen tumors were pulverized on dry ice using a small Bessman tissue pulverizer (Spectrum Laboratories, Rancho Dominguez, Calif.) and protein extracts were prepared at 4° C. with Cell Extraction Buffer (Invitrogen, Carlsbad, Calif.), 1 mM phenylmethylsulphonyl fluoride (PMSF), Phosphatase Inhibitor Cocktail 1/2 (Sigma-Aldrich, St. Louis, Mo.), and one tablet of Complete EDTA-free Mini™ protease inhibitor cocktail (Roche Diagnostics, Indianapolis, Ind.). Lysates were subjected to centrifugation at 16,100 g for 15 minutes and protein concentration was determined using the BCA protein assay (Pierce Biotechnology, Rockford, Ill.). For Western blot analysis, proteins were resolved by 4-12% SDS-PAGE and transferred to nitrocellulose membranes (Millipore Corporation, Billerica, Mass.). Immunoblotting was performed using primary antibodies for PAK1, p27, E2F1 (Cell Signaling Technology, Danvers, Mass.) and anti-β-actin (Sigma-Aldrich, St. Louis, Mo.). Secondary antibodies were obtained from Pierce Biotechnology (Rockford, Ill.).

Immunoflourescence imaging was performed using primary antibodies for p27^{Kip1} (Becton Dickinson, San Jose, Calif.). Secondary antibodies were obtained from Millipore Corporation (Billerica, Mass.). Images were analyzed in Metamorph (version 7.5.3.0, MDS Analytical; Sunnyvale, Calif.) using an automated analysis routine. Briefly, a smoothing filter was applied to the DAPI channel to even out the nuclear staining pattern. The MWCS application in Metamorph was then used to identify and count DAPI stained nuclei, and classify them as positive or negative for p27 in the Cy3 channel.

### EXAMPLE 6

### Tumor Xenograft Models

Cultured NCI-H520.X1 and EBC1 cells were removed from culture, suspended in Hank's buffered saline solution (HBSS), mixed 1:1 with Matrigel (BD Biosciences, USA), and implanted subcutaneously into the right flank of naïve female NCR nude mice (Taconic Farms, Hudson, N.Y.). Mice with tumors of a mean volume of approximately 250 mm³ were grouped into treatment cohorts of 10 mice each. Mice received 5% sucrose only or 5% sucrose plus 1 mg/ml doxycycline (Clontech, Mountain View, Calif.) for control and knockdown cohorts, respectively. All water bottles were changed 3 times per week. Body weights and tumor volume measurements (as obtained by length and width measurements with calipers) were taken twice per week during the study. All experimental procedures conformed to the guiding principles of the American Physiology Society and were approved by Genentech's Institutional Animal Care and Use Committee: Tumor volumes were calculated by the following formula: Tumor Volume=0.5*(a*b²), where ‘a’ is the largest tumor diameter and ‘b’ is the perpendicular tumor diameter. Tumor volume results are presented as mean tumor volumes±the standard error of the mean (SEM). Percent growth inhibition (% INH) at the end of study (EOS) was calculated as % INH=100 [(EOS Vehicle−EOS Treatment)/(EOS Vehicle)]. Data analysis and generation of p-values using the Dunnett t-test was done using JMP software (SAS Institute, Cary, N.C.).

Xenograft tissues were fixed for 24 h in 10% neutral buffered formalin and were then processed and paraffin embedded. Sections were cut at a thickness of 3 μm, and specimens with sufficient viable tumor (assessed on H&E-stained slides) were further evaluated by immunohistochemistry. Anti-Ki-67 (clone MIB-1, mouse anti-human) was used with the DAKO ARK Kit for detection. Tissues were counterstained with hematoxylin, dehydrated, and mounted. Antigen retrieval was done with the DAKO Target Retrieval Kit as per manufacturer's instructions. For quantification of immunohistochemically Ki-67-positive cells, images were acquired by the Ariol SL-50 automated slide-scanning platform (Genetix Ltd.) at ×100 final magnification. Tumor-specific areas were identified manually for analysis in the Ariol software. A 3,3′-diaminobenzidine-specific color range was specified using the hue, saturation, and intensity color space to quantify the area of staining and the output was total Ki-67-positive cells in relation to total cell count.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

1-36. (canceled)

37. A method for treating tumors comprising contacting said tumor with a PAK1 inhibitor in combination with a second anti-hyperproliferative compound.

38. The method according to claim 37 wherein said tumor exhibits elevated levels of the PAK1 protein.

39. The method according to claim 37 wherein said tumor is breast tumor, a squamous non-small cell lung tumor or a squamous head and neck tumor.

40. The method according to claim 37 wherein the PAK1 inhibitor is a compound of formula I:

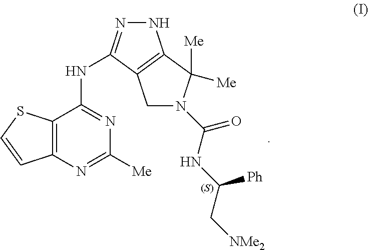

41. The method of any of claim 37 wherein said the second anti-hyperproliferative compound is an inhibitor of inhibitor of apoptosis proteins.

42. The method of claim of claim 41 wherein said inhibitor of inhibitor of apoptosis protein is BV6 or G24416.

43. The method of any of claim 37 wherein said anti-hyperproliferative compound is an EGFR inhibitor or antagonist.

44. The method of claim 43 wherein said EGFR inhibitor is erlotinib, gefitinib or lapatinib.

45. The method of any of claim 37 wherein said second anti-hyperproliferative compound is an inhibitor of the Ras/Raf/MEK/Erk signaling cascade.

46. The method of any of claim 37 wherein said second anti-hyperproliferative compound is an inhibitor of PI3K/AKT/mTOR signaling cascade.

47. The method of any of claim 37 wherein the second anti-hyperproliferative compound is an inhibitor of Src kinase.

48. A method of treating a patient suffering from a cancer or a hyperproliferative disorder comprising co-administering to a patient in need thereof a PAK 1 inhibitor and a second anti-hyperproliferative agent.


SEQUENCE LISTING (6 sequences)

SEQ ID NO: 1 - DNA, 90 bp, Escherichia coli
cttataagttccctatcagtgatagagatccccaataagcgttggcaatttattcaagagataaattgccaacgcttattttttttggaa

SEQ ID NO: 2 - DNA, 90 bp, Escherichia coli
ttccaaaaaaaataagcgttggcaatttatctcttgaataaattgccaacgcttattggggatctctatcactgatagggaacttataag

SEQ ID NO: 3 - DNA, 65 bp, Homo sapiens
gatccccgaagagaggttcagctaaattcaagagatttagctgaacctctcttcttttttggaaa

SEQ ID NO: 4 - DNA, 65 bp, Homo sapiens
agcttttccaaaaaagaagagaggttcagctaaatctcttgaatttagctgaacctctcttcggg

SEQ ID NO: 5 - DNA, 65 bp, Homo sapiens
gatccccctgcataacctgaatgaaattcaagagatttcattcaggttatgcagttttttggaaa

SEQ ID NO: 6 - DNA, 65 bp, Homo sapiens
agcttttccaaaaaactgcataacctgaatgaaatctcttgaatttcattcaggttatgcagggg